(19) United States
(12) Patent Application Publication
(10) Pub. No.: US 2014/0128652 A1
(43) Pub. Date: May 8, 2014

(54) METHOD FOR PRODUCING DIISOBUTYLENE USING MIXED C4 FRACTION AS RAW MATERIAL

(71) Applicants: Fumio Yamakawa, Chiba (JP); Kei Tatesaki, Chiba (JP); Takashi Nakagawa, Chiba (JP)

(72) Inventors: Fumio Yamakawa, Chiba (JP); Kei Tatesaki, Chiba (JP); Takashi Nakagawa, Chiba (JP)

(73) Assignee: IDEMITSU KOSAN CO., LTD., Chiyoda-ku, Tokyo (JP)

(21) Appl. No.: 14/129,410

(22) PCT Filed: Jun. 6, 2012
(86) PCT No.: PCT/JP2012/064590

§ 371 Date: Dec. 26, 2013

(30) Foreign Application Priority Data
Jun. 29, 2011 (JP) 2011-144900

(51) Int. Cl. C07C 2/06

(52) U.S. Cl. CPC C07C 2/06
USPC 585/510

(57) ABSTRACT

There is provided a process for producing high-purity diisobutylene with a high reaction selectivity in which a mixed C4 fraction is contacted with an oligomerization catalyst to subject isobutene to oligomerization at one stage. The present invention relates to a process for producing diisobutylene by contacting a mixed C4 fraction as a raw material with a solid acid catalyst, which includes the steps of (a) isobutene oligomerization; (b) subjecting the resulting reaction products to distillation to separate the unreacted C4 fraction and an oligomer fraction including a C8 fraction produced from each other; and (c) purification of diisobutylene from the C8 fraction by distillation, in which a conversion of isobutene contained in the mixed C4 fraction upon conducting the step (a) is controlled to a range of from 60 to 95%.

### TECHNICAL FIELD

The present invention relates to a process for producing diisobutylene using a mixed C4 fraction as a raw material, and more particularly, to a process for producing high-purity diisobutylene having a high added value with a high reaction selectivity in which the mixed C4 fraction is contacted with a solid acid catalysts, preferably a silica-alumina catalyst to subject isobutene to oligomerization at one stage, and then the resulting reaction products are subjected to distillation process.

### BACKGROUND ART

It is known that diisobutylene as dimer of isobutene (hereinafter occasionally referred to merely as “DIB”) is useful as a raw material for oxoalcohols, isononanoic acid, p-octyl phenol, rubber tackifiers, surfactants, gasoline fuel additives, rubber chemicals or the like.

Diisobutylene may be generally produced by the method in which isobutene derived from a C4 fraction produced in FCC (Fluid Catalytic Cracking) plant or ethylene production plant is selectively reacted with sulfuric acid to obtain isobutyl sulfate and separate the resulting product from 1-butene, 2-butene, butane, etc., and then the thus obtained isobutyl sulfate is subjected to heat-cracking to thereby obtain the diisobutylene as aimed. Alternatively, diisobutylene may be produced by the method in which MTBE (methyl tert-butyl ether) or TBA (tert-butyl alcohol) is obtained from isobutene derived from a C4 fraction, and then the thus obtained MTBE or TBA is subjected to cracking or dimerization to produce diisobutylene as aimed.

In the former production method, there tends to occur such problems that a large amount of oligomers such as trimers and tetramers or higher polymers are by-produced in addition to the aimed diisobutylene, resulting in low reaction selectivity of DIB, and expensive anti-corrosive materials are required therefor. Further, in both the production methods, there tends to occur such a problem that a large number of the complex reaction steps are required.

On the other hand, Patent Document 1 discloses the method of oligomerizing isobutene using acid ion exchange resin having a sulfonic group in which protons of the acid ion exchanger are partially replaced with a metal ion. In this method, diisobutylene having a high purity is produced from a mixed C4 fraction as a raw material. However, the conversion of isobutene in the method is low, and the oligomerization catalyst used therein is quite different from a silica-alumina catalyst.

Also, Patent Document 2 discloses the method of oligomerizing olefins in which fuels, for example, gasoline and/or kerosene/light gas oil (diesel) can be produced using a silica-alumina catalyst. In this method, a mixed C4 fraction as a raw material is oligomerized with 90% or more conversion of isobutene, or with about 90% conversion of 1-butene and about 80% conversion of 2-butene. However, the aimed product of the above method is C5 or higher polymer products (there is no description concerning a purity of diisobutylene). In addition, in Examples of Patent Document 2, it is described that the conversion of isobutene was in the range of from 97 to 100%, i.e., isobutene was reacted in a region where the conversion thereof is extremely high.

### PRIOR DOCUMENTS

### Patent Documents

Patent Document 1: JP-A 2004-123714

Patent Document 2: JP-A 2006-28519

### SUMMARY OF THE INVENTION

### Problems to be Solved by the Invention

The present invention has been accomplished in view of the above problems encountered in the art, and an object of the present invention is to provide a process for producing high-purity DIB with a high reaction selectivity in which a mixed C4 fraction is contacted with an oligomerization catalyst to subject isobutene to oligomerization at one stage.

### Means for Solving the Problems

As a result of intensive and extensive researches for achieving the above object, the present inventors have found that when a mixed C4 fraction is contacted with a solid acid catalyst, preferably a silica-alumina catalyst, as an oligomerization catalyst while controlling a conversion of isobutene in the mixed C4 fraction to a desired range, it is possible to produce DIB with a high reaction selectivity, and further that when subjecting the obtained reaction product to a specific distillation process, it is possible to obtain DIB having high purity.

The present invention has bee accomplished on the basis of this finding.

That is, the present invention relates to the following aspects.

[1] A process for producing diisobutylene by contacting a mixed C4 fraction as a raw material with a solid acid catalyst, including the steps of;

(a) isobutene oligomerization;

(b) subjecting the resulting reaction products to distillation to separate the unreacted C4 fraction and an oligomer fraction including a C8 fraction produced from each other; and

(c) purification of diisobutylene from C8 fraction by distillation, in which a conversion of isobutene contained in the mixed C4 fraction upon conducting the step (a) is controlled to a range of from 60 to 95%.

[2] The process for producing diisobutylene as described in the above aspect [1], wherein the solid acid catalyst is a silica-alumina catalyst.
[3] The process for producing diisobutylene as described in the above aspect [19 or [2], wherein the step (a) of isobutene oligomerization is carried out under reaction conditions including WHSV (mass of feed stock based on mass of catalyst per 1 h) of the mixed C4 fraction on the basis of the solid acid catalyst of from 0.1 to 5 h⁻¹, a reaction temperature of 150° C. or lower, and a reaction pressure of 0.2 MPa or more at which the raw material is liquefiable.
[4] The process for producing diisobutylene as described in any one of the above aspects [1] to [3], wherein in the step (c) of purification of diisobutylene from C8 fraction by distillation, the diisobutylene obtained therein has a C4 fraction content of 1% by mass or less and a purity of 95% by mass or more.

### EFFECT OF THE INVENTION

In the process for producing DIB according to the present invention, by contacting a mixed C4 fraction with a solid acid catalyst, preferably a silica-alumina catalyst, as an oligomerization catalyst while controlling a conversion of isobutene in the mixed C4 fraction to a desired range and further subjecting the obtained reaction product to a specific distillation process, it is possible to produce high-purity DIB with a high reaction selectivity in an efficient manner.

### PREFERRED EMBODIMENTS FOR CARRYING OUT THE INVENTION

The process for producing DIB according to the present invention in which a mixed C4 fraction as a raw material is contacted with a solid acid catalyst, is characterized by including the steps of;

(a) isobutene oligomerization;

(b) subjecting the resulting reaction products to distillation to separate the unreacted C4 fraction and an oligomer fraction including a C8 fraction produced from each other; and

(c) purification of diisobutylene from C8 fraction by distillation, in which a conversion of isobutene contained in the mixed C4 fraction upon conducting the step (a) is controlled to a range of from 60 to 95%.

### [Mixed C4 Fraction]

In the process for producing DIB according to the present invention, the mixed C4 fraction is used as a raw material therefor.

Examples of the mixed C4 fraction include olefin fractions obtained in FCC process, olefin fractions obtained by removing diene compounds from fractions produced in naphtha cracker by extraction or selective hydrogenation thereof, and mixtures obtained by mixing these fractions at an optional ratio. Further, as the mixed C4 fraction, there may also be used those prepared by increasing or reducing the contents of any specific compounds in these fractions by conventionally known methods such as distillation. For example. there may be used a raffinate obtained by extracting butadiene from a C4 fraction produced in naphtha cracker, and an isobutene-isobutane fraction having high isobutene concentration which is obtained by subjecting a C4 fraction produced in FCC process to distillation (or reaction distillation) to remove n-butenes or n-butanes therefrom.

The mixed C4 fraction generally contains various compounds such as 1-butene, trans-2-butene, cis-2-butene, isobutene, n-butane, isobutane and butadiene.

### (Pretreatment of Mixed C4 Fraction)

In the process for producing DIB according to the present invention, the mixed C4 fraction as a raw material thereof is preferably subjected to the following pretreatments (1) to (3) to remove impurities and purify the raw material.

(1) Dienes such as butadiene in the mixed C4 fraction which tend to cause deactivation in catalytic activity or decrease in purity of diisobutylene may be removed using an extraction by solvent such as N,N-dimethyl formamide and acetonitrile. Further, the content of the dienes in the mixed C4 fraction may also be reduced by selective hydrogenation using a hydrogenation catalyst such as Pd or Ni, if required. In general, the content of the dienes in the fraction is suitably adjusted to 1000 ppm by mass or less.
(2) Sulfur compounds and/or basic nitrogen compounds which tend to cause deactivation in catalytic activity may be removed by washing with water, treatments with adsorbing agents such as activated alumina, activated carbon and/or molecular sieves.
(3) The C3 fraction which tends to cause decrease in purity of diisobutylene may be removed by distillation from a top of a distillation column.

### [(a) Step of Isobutene Oligomerization]

In the process for producing DIB by contacting the mixed C4 fraction as a raw material thereof which is preferably subjected the above pretreatments with a solid acid catalyst according to the present invention, the conversion of isobutene in the mixed C4 fraction upon conducting the step (a) of isobutene oligomerization is controlled to a range of from 60 to 95%.

When the conversion of isobutene in the mixed C4 fraction is more than 95%, not only 2,4,4-trimethyl-1-pentene and 2,4,4-trimethyl-2-pentene as the aimed diisobutylene but also heavy compounds such as C8 isomers obtained by the reaction of 1-butene or 2-butene as well as trimers or tetramers thereof are produced in an increased amount, so that the subsequent distillation procedure may fail to produce diisobutylene having a high purity. When the conversion of isobutene in the mixed C4 fraction is less than 60%, the amount of the unreacted raw materials in the resulting diisobutylene tends to be increased, so that it may be difficult to produce diisobutylene with a high yield. From the above viewpoints, the conversion of isobutene is preferably from 65 to 90% and more preferably from 70 to 90%.

Meanwhile, the method of controlling a conversion of isobutene is described in detail below.

### (Solid Acid Catalyst)

Examples of the solid acid catalyst used as the oligomerization catalyst in the process for producing DIB according to the present invention include silica-alumina, silica-magnesia, silica-boria, alumina-boria, chlorinated alumina, fluorinated alumina, those obtained by fixing hydrochloric acid, sulfuric acid, phosphoric acid or BF₃ onto silica gel or alumina gel, cation exchange resins, synthetic zeolites, heteropolyacids, zirconia-based composite metal oxides such as molybdenum oxide/zirconia and tungsten oxide/zirconia, and clay minerals such as acid clay, bentonite, kaolin and montmorillonite. These solid acid catalysts may be used alone or in combination of any two or more thereof. Among these solid acid catalysts, especially preferred is silica-alumina.

The silica-alumina catalyst may be produced by a deposition method in which alumina is deposited on silica gel using an Al₂(SO₄)₃ solution and NH₄OH, or a precipitation method in which a sodium silicate (water glass) solution is added to an Al₂(SO₄)₃ solution. When calcining the thus obtained gel at a temperature of about 550° C., it is possible to obtain a catalyst having an extremely high activity.

As the silica-alumina catalyst, there may be generally used those having the following properties:

Mass ratio of SiO₂/Al₂O₃: 2 to 20;

Average pore diameter as measured by nitrogen adsorption method: 2 to 10 nm;

Total pore volume as measured by nitrogen adsorption method: 0.2 to 1 mL/g; and

BET specific surface area: 200 to 600 m²/g.

### (Method of Controlling Conversion of Isobutene)

The conversion of isobutene may be controlled under the following reaction conditions.

(1) The WHSV (mass of feed stock based on mass of catalyst per 1 h) of the mixed C4 fraction on the basis of the solid acid catalyst is preferably from 0.1 to 5 h⁻¹ and more preferably from 0.2 to 2 ⁻¹. When the WHSV is 0.1 h⁻¹ or more, it is possible to maintain the conversion of isobutene 95% or less. On the other hand, when the WHSV is 5 h⁻¹ or less, it is possible to maintain the conversion of isobutene 60% or more.
(2) The reaction temperature is preferably 150° C. or lower and more preferably from 25 to 100° C. When the reaction temperature is 25° C. or higher, it is possible to achieve an adequate reaction rate without using a large amount of the catalyst. On the other hand, when the reaction temperature is 150° C. or lower, it is possible to suppress decrease in reaction selectivity of DIB, so that DIB having a high purity can be obtained.
(3) The reaction pressure is preferably 0.2 MPa or more as long as the raw material may be liquefied thereunder.
(4) The reaction may be carried out using an adiabatic reactor, a multipipe reactor or the like. In order to control the reaction temperature (removal of heat), the reaction products may be recycled to the reactor (feeding in the form of a mixture with the raw material), or the raw material may be diluted with any suitable diluent. When recycling the reaction products to the reactor, the amount of the reaction products to be recycled is preferably from 0 to 4 times by mass and more preferably from 0 to 3 times by mass the amount of the raw material fed to the reactor. When the amount of the reaction products to be recycled is 4 times by mass or less, it is possible to prevent decrease of a reaction rate owing to decrease in concentration of the raw material, so that use of a large amount of the catalyst is not needed.

Meanwhile, the reactor and reaction method used upon the oligomerization reaction of isobutene are not particularly limited, and there may be adopted batch-type, semi-batch-type and continuous flow-type reaction methods using vessel-type reactor, continuous flow-type reaction methods using fixed bed, fluidized bed or moving bed flow reactor, etc.

The process for producing DIB according to the present invention further includes the step (b) of subjecting the resulting reaction products to distillation to separate the unreacted C4 fraction and an oligomer fraction including a C8 fraction produced from each other, and the step (c) of purification of DIB from C8 fraction by distillation.

The step (b) is such a step in which the reaction products obtained in the oligomerization reaction is subjected to distillation to separate the unreacted C4 fraction and an oligomer fraction including a C8 fraction produced which are contained in the reaction products, from each other.

In the step (b), the reaction products are fed to distillation column, and subjected to distillation operation while controlling the distillation conditions (such as a reflux ratio (R/D), a pressure, etc.) such that the content of a C4 fraction in the bottoms is 1% by mass or less. As a result, the C4 fraction as the unreacted raw material is removed from a top of the column, whereas oligomers including diisobutylene in a C8 fraction are obtained as the bottoms.

[Step (c)1]

The step (c) is such a step in which diisobutylene in the C8 fraction obtained in the step (b) is purified by distillation. In the step (c), the bottoms including diisobutylene which are obtained in the step (b) are fed to a distillation column, and the C8 fraction mainly containing diisobutylene is obtained from a top of the column, whereas oligomer components mainly containing trimer or higher oligomers are obtained from a bottom of the column.

In the step (c), it is possible to obtain diisobutylene having a C4 fraction content of 1% by mass or less and a purity of 95% by mass or more.

### Use of Diisobutylene]

Diisobutylene (2,4,4-trimethyl-1-pentene and 2,4,4-trimethyl-2-pentene) produced by the process for producing DIB according to the present invention may be used, for example, in the applications including a raw material for oxoalcohols, isononanoic acid, p-octyl phenol, rubber tackifiers, surfactants, gasoline fuel additives, rubber chemicals or the like.

### EXAMPLES

The present invention will be described in more detail below with reference to the following examples. However, these examples are only illustrative and not intended to limit the invention thereto.

### Examples 1 to 3 and Comparative Example 1

A diene compound such as butadiene in a mixed C4 fraction obtained from cracking of naphtha was extracted therefrom with dimethyl formaldehyde, and then the remaining C4 fraction was subjected to selective hydrogenation using a commercially available Pd catalyst such that a concentration of the diene compound therein was 10 ppm by mass or less. Successively, the resulting reaction product was washed with water to remove sulfur (S) compounds and nitrogen (N) compounds therefrom until a concentration of the respective compounds was reduced to 5 ppm by mass or less. Further, the thus washed product was subjected to continuous distillation to remove C3 compounds from top of the column and use the remaining reaction liquids containing the C4 fraction as a raw material for production of diisobutylene (the composition of the raw material is shown in Table 1).

A silica-alumina catalyst [mass ratio SiO₂/Al₂O₃: 9; average pore diameter: 6 nm; total pore volume: 0.5 mL/g; BET specific surface area: 400 m²/g] as an oligomerization catalyst was packed in a tubular reactor to conduct a fixed-bed continuous reaction therein. The reactor thus packed with the catalyst was heated by a mantle heater such that a catalyst bed therein was controlled in an isothermal condition. The reaction conditions and the reaction results are shown in Table 1.

In Examples 1 to 3, the reaction pressure and WHSV were respectively fixed to specific values, whereas the reaction temperature was varied, whereby the conversion of isobutene was adjusted to less than 95%. On the other hand, in Comparative Example 1, the reaction pressure and WHSV were respectively fixed to specific values similarly to Examples 1 to 3, and the reaction temperature was adjusted to 80° C., whereby the conversion of isobutene was adjusted to 98%.

Further, the resulting reaction products was subjected to continuous distillation under the conditions including 18 distillation column stages, a reflux ratio R/D of 0.29 and a column top pressure of 0.38 MPa to remove the C4 compounds as the unreacted raw material therefrom. Successively, the bottoms including diisobutylene as feed liquid were subjected to continuous distillation under the conditions including 40 distillation column stages, a reflux ratio R/D of 5 and a column top pressure of 0.65 MPa to obtain C8 compounds containing diisobutylene from top of the column. The concentrations of 2,4,4-trimethyl-1-pentene and 2,4,4-trimethyl-2-pentene as the aimed diisobutylene in the thus obtained C8 compounds are shown in lowermost column of Table 1.

TABLE 1

| | | Com. | Examples | | |
|---|---|---|---|---|---|
| | | Ex. 1 | 1 | 2 | 3 |
| Composition of raw material | mol % | | | | |
| Isobutene | | 45 | 45 | 45 | 47 |
| n-Butenes | | 40 | 39 | 38 | 39 |
| Butanes | | 14 | 15 | 16 | 13 |
| C3 compounds | | 0.03 | 0.03 | 0.02 | 0.03 |
| Dienes | | 0.000 | 0.000 | 0.000 | 0.001 |
| Reaction temperature | ° C. | 80 | 68 | 61 | 37 |
| Reaction pressure | MPa | 2 | 2 | 2 | 2 |
| WHSV | h⁻¹ | 0.2 | 0.2 | 0.2 | 0.2 |
| Isobutene conversion | % | 98 | 93 | 86 | 74 |
| n-Butene conversion | % | 13 | 8 | 5 | 2 |
| Composition of reaction | | | | | |
| product liquid | mass % | | | | |
| C8 | | 40 | 46 | 54 | 58 |
| C12 | | 49 | 47 | 41 | 36 |
| C16 | | 9 | 6 | 4 | 4 |
| C20+ | | 2 | 2 | 1 | 2 |
| Concentration of DIB in C8 | | 66 | 80 | 86 | 93 |
| compounds | | | | | |
| Concentration of DIB after | mass % | 90 | 95 | 96 | 97 |
| purification by distillation | | | | | |

From the results shown in Table 1, it was confirmed that in Examples 1 to 3 in which the conversion of isobutene was in the range of from 74 to 93%, it was possible to obtain a high-purity product having a diisobutylene concentration of 95% or more. On the other hand, it was confirmed that in Comparative Example 1 in which the conversion of isobutene was 98%, diisobutylene obtained therein had a purity of 90%.

### INDUSTRIAL APPLICABILITY

In the process for producing DIB according to the present invention, when a mixed C4 fraction is contacted with a solid acid catalyst, preferably a silica-alumina catalyst to subject isobutene to oligomerization at one stage and then the obtained reaction products are subjected to distillation process, it is possible to produce high-purity diisobutylene having a high added value with a high reaction selectivity.

1. A process for producing diisobutylene by contacting a mixed C4 fraction as a raw material with a solid acid catalyst, the process comprising:
performing isobutene oligomerization to produce reaction products;
subjecting the reaction products to distillation to separate unreacted C4 fraction and an oligomer fraction comprising a C8 fraction produced from each other; and
purifying diisobutylene from the C8 fraction by distillation,
in which a conversion of isobutene in a mixed C4 fraction upon conducting the oligomerization is controlled to a range of from 60 to 95%.

2. The process according to claim 1, wherein the solid acid catalyst is a silica-alumina catalyst.

3. The process according to claim 1, wherein the performing of isobutene oligomerization is carried out under reaction conditions comprising WHSV, mass of feed stock based on mass of catalyst per 1 h, of the mixed C4 fraction on a basis of the solid acid catalyst of from 0.1 to 5 h⁻¹, a reaction temperature of 150° C. or lower, and a reaction pressure of 0.2 MPa or more at which the raw material is liquefiable.

4. The process according to claim 1, wherein in the purification of diisobutylene from the C8 fraction by distillation, the diisobutylene obtained therein has a C4 fraction content of 1% by mass or less and a purity of 95% by mass or more.
